(19) United States
(12) Patent Application Publication
(10) Pub. No.: US 2022/0354654 A1
(43) Pub. Date: Nov. 10, 2022

(54) SACROILIAC JOINT FUSION SYSTEM

(71) Applicants: Adam Isaac Lewis, Madison, MS (US); Lauren Chase Thornburg, Cumming, GA (US)

(72) Inventors: Adam Isaac Lewis, Madison, MS (US); Lauren Chase Thornburg, Cumming, GA (US)

(21) Appl. No.: 17/735,359

(22) Filed: May 3, 2022

Related U.S. Application Data
(60) Provisional application No. 63/184,881, filed on May 6, 2021.

(51) Int. Cl. A61F 2/30

(52) U.S. Cl. CPC A61F 2/30988; A61F 2002/30622; A61F 2002/30995; A61F 2002/30858; A61F 2002/2835

(57) ABSTRACT

A sacroiliac joint screw has a screw body having a head and a treaded shank with a self-drilling cutting tip. The screw body is cannulated and configured to receive a Steinmann pin for the purpose of a minimally invasive approach (MIS) for delivery to the sacroiliac joint (SI) while minimizing soft tissue damage. The self-drilling cutting tip creates a pilot hole. The threaded shank has a plurality of spiral cutting flutes, the spiral cutting flutes extend along a length of the threaded shank and are configured to provide a constant self-tapping feature. A plurality of bone harvesting windows are positioned in the spiral cutting flutes and are configured to pull in bone as the screw is advanced into the joint.

### FIELD OF THE INVENTION

The present invention relates to a Sacroiliac Joint Fusion System intended for large bone fixation, including sacroiliac joint fusion for conditions including sacroiliac joint disruptions and degenerative sacroiliitis.

### BACKGROUND OF THE INVENTION

The sacrum is a large triangular-shaped bone found at the base of the spinal column. It consists of the last four or five vertebrae that by adulthood, fuse together to form a single bone. Located just above the coccyx and wedged between the right and left iliac bones (hip bones), the sacrum forms the back wall of the pelvis. The coccyx, commonly referred to as the tailbone, is the very bottom segment of the vertebral column. The right and left iliac bones are joined together in the front by the symphysis pubis (a small joint that connects the pubic bones).

The sacrum is slightly curved, giving the pelvic cavity more room to house organs and tissue. Interestingly, the sacrum is shorter and wider in females than in males. The term for such differences between males and females of the same species is sexual dimorphism. This is why men tend to have a narrow pelvic cavity, and women, a more hourglass shape for bearing children.

The top of the sacrum is connected to the lumbar vertebrae and the base is joined to the coccyx. Wings on either side of the bone are called the sacral ala. These wings fit similar to interlocking pieces of a puzzle between the two halves of the pelvis. It is this junction of the sacral alae and the iliac bones that forms the sacroiliac joint as described hereinafter.

In vertebrate anatomy, the sacrum is a large, triangular bone that lies at the bottom of the lumbar spine, where it connects with the L5 vertebra. The sacrum lies adjacent to two hip bones, known as the right ilium and left ilium. The sacrum connects with these bones via joints known as sacroiliac joints (or SI joints).

The sacroiliac joints assist in the transmission of forces from the spine to the lower extremities. Degeneration of the sacroiliac joints can occur due to diseases, such as degenerative sacroiliitis and inflammatory sacroiliitis, as well as due to normal aging and trauma. One type of treatment for a degenerated sacroiliac joint is fusion of the joint, which ultimately relieves pain.

Thus, there remains a need for improved implants that assist in the fusion of sacroiliac joints.

### SUMMARY OF THE INVENTION

A sacroiliac joint screw has a screw body having a head and a threaded shank with a self-drilling cutting tip. The screw body is cannulated and configured to receive a Steinmann pin for the purpose of a minimally invasive approach (MIS) for delivery to the sacroiliac joint (SI) while minimizing soft tissue damage. The self-drilling cutting tip creates a pilot hole. The threaded shank has a plurality of spiral cutting flutes, the spiral cutting flutes extend along a length of the threaded shank and are configured to provide a constant self-tapping feature. A plurality of bone harvesting windows are positioned in the spiral cutting flutes and are configured to pull in bone as the screw is advanced into the joint.

The sacroiliac joint screw further may have an integrated removable tower for soft tissue protection. The tower is fixed or otherwise attached at a proximal end of the screw. The screw head has a reverse threaded external head geometry configured to allow a rescue tower to attach to the screw for screw removal. The reverse threaded external head geometry is a left-handed thread which allows loosening of the screw. The screw head has an undercut on a distal side of the head. The undercut allows for a stab and grab attachment to a driver. The screw has a secure threaded post-fill bone graft delivery tube mechanism to provide bone graft material.

The sacroiliac joint screw may further have a driver with a hexalobular drive configured to be received in an hexalobular drive aperture in the screw head. The screw is preferably made of a titanium alloy in diameter sizes of 8.0 mm to 12.0 mm and made in lengths from 25 mm to 60 mm.

The sacroiliac joint screw may further include a load dispersion cap attached to an underside of the screw head. The cap disperses the surface load and is configured to pivot and spin underneath the screw head.

A method of sacroiliac joint fusion comprises the steps of: localizing the sacroiliac joint by fluoroscopy; inserting the 2.8 mm Steinmann Pin to the plane of the SI joint; sliding Dilator One over the Steinmann Pin; sliding the Dilator Two over Dilator One; sliding Dilator Three over Dilator Two; removing Dilator One and inserting the Screw Depth Gauge over the Steinmann Pin; reading the depth marking closest to the top of the Steinmann Pin; verifying the depth via fluoroscopy; removing the Screw Depth Gauge and Dilator Two to prepare for drilling; selecting the desired Drill Bit size and drill to the depth reading indicated from the Screw Depth Gauge; attaching the appropriately sized SI Screw to the physicians preferred Driver and insert over the Steinmann Pin; threading until fully seated, the Load Dispersion Cap should be threaded over the distal end of SI Screw prior to implantation if one is to be used; removing Dilator Three and inserting the 14 mm or 16 mm Parallel Pin Guide over the Steinmann Pin; using fluoroscopic imaging to align the Parallel Pin Guide; inserting the second Steinmann Pin and repeating the steps above for any remaining SI Screw; threading in the Graft Filler Tube to the SI Screw and using the Graft Tamp to deliver any allograft or autograft of the physician's choice to the SI Screw cannulation; and removing all instrumentation and snapping the extension towers off the SI screws via a rocking motion.

### BRIEF DESCRIPTION OF THE DRAWINGS

The invention will be described by way of example and with reference to the accompanying drawings in which:

FIG. 1 is a plan view of the sacroiliac joint screw of the present invention

FIG. 2 is a plan view of the sacroiliac joint screw from FIG. 1 without the breakaway tower.

FIG. 3 is a top view of the sacroiliac joint screw of FIG. 2.

FIG. 4 is an illustration of step 1 of an exemplary sacroiliac joint fusion procedure using a Steinmann pin in combination with a schematic view of fluoroscopic imaging to determine initial trajectory to the SI joint.

FIG. 5 is an illustration of step 2 showing dilator one passing over the Steinmann pin.

FIG. 6 is an illustration of step 3 passing dilator two over dilator one.

FIG. 7 is an illustration of step 4 passing dilator three over dilator two.

FIG. 8 is an illustration of step 5 removing Dilator One and inserting the Screw Depth Gauge over the Steinmann Pin and advancing the Depth Gauge until it contacts bone distally.

FIG. 9 is an illustration of step 6 showing the screw depth gauge for reading the number at the top of the Steinman pin.

FIG. 10 is an illustration of step 7 showing an exemplary drill used with the Quick Connect Palm handle or drill motor, the drill can be one of 5 mm, 7 mm or 9 mm.

FIG. 11 is an illustration of step 8 advancing the Drill Bit to the corresponding number read from the Screw Depth Gauge.

FIG. 12 is an illustration of step 9 threading the appropriately sized SI Screw onto the Driver Assembly.

FIG. 13 is an illustration of step 10 advancing the SI Screw over the Steinmann Pin and threading into the prepared bone hole until tight.

FIG. 14 is an illustration of step 11 wherein the Driver Assembly was removed by rotating the knurled knob shown in FIG. 13 counterclockwise leaving the dilators three and two and the SI screw.

FIG. 15 is an illustration of step 12 removing Dilator Three leaving behind the Steinmann Pin.

FIG. 16 is an illustration of step 13 placing the preferred sized Parallel Pin Guide over the Initial Steinmann Pin.

FIG. 17 is an illustration of step 14 using fluoroscopic imaging to align the Parallel Pin Guide in the correct orientation and deliver a second Steinmann Pin through the open cannula for an additional SI screw placement.

FIG. 18 is an illustration of step 15 advancing the Steinmann Pin to the desired depth and step 16 Remove the Parallel Pin Guide and first Steinmann Pin from the initial SI Screw.

FIG. 19 is an illustration of step 17 filling the Graft Filler Tube with the surgeon's preferred autograft or allograft (not shown).

FIG. 20 is an illustration of step 18 loading the Graft Filler Tamp on the quick connect handle.

FIG. 21 is an illustration of step 19 advancing the Tamp deploying the bone graft into the SI Screw and surrounding bone.

FIG. 22 is an illustration of step 20 with the towers removed from the SI Screws.

FIG. 23 is a side view of FIG. 22.

FIGS. 24A, 24B and 24C are exemplary illustrations of a surgical tray or kit for SI joint fusion, each figure showing a tray layer included in the kit or set.

FIG. 25 is an illustration of an exemplary fluoroscope which is used to image the SI joint for optimum pin or screw placement.

### DETAILED DESCRIPTION OF THE INVENTION

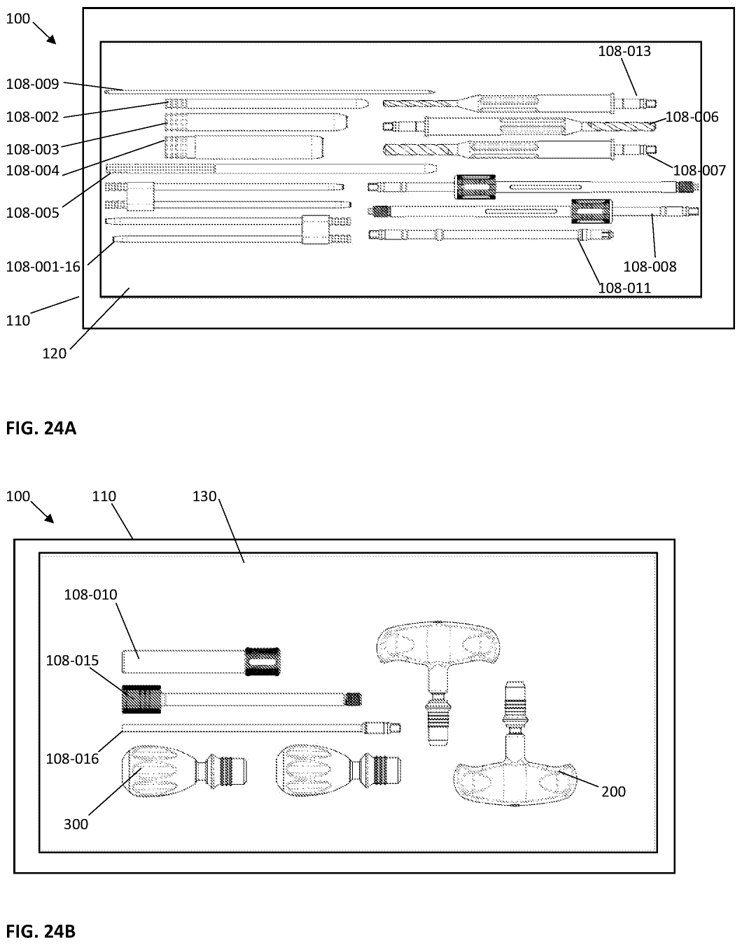

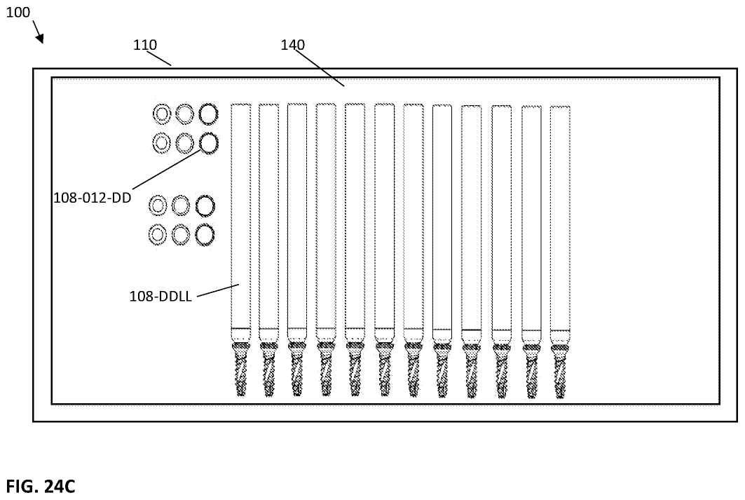

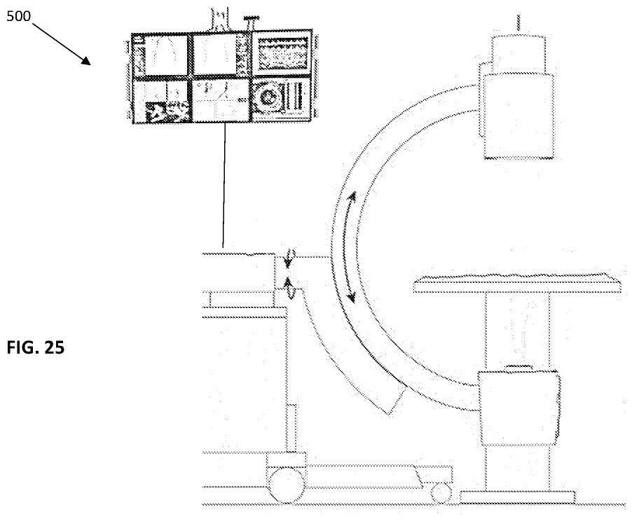

The instrumentation provided is focused on site approach, access, tissue preparation, screw delivery, and bone graft delivery. This set or kit 100, as shown in FIGS. 24A. 24B and 24C, is designed as a full solution for fusion surgery of the sacroiliac (SI) joints. The main surgical tray 110 is shown with three layers of trays 120, 130 and 140. Some key instruments provided include Steinmann pins 108-009, pin guides 108-001-14, 108-001-16, sequential dilators 108-002, 108-003, 108-004, and an access portal shown in FIG. 24A tray layer 120. The purpose of these instruments is to determine surgical site approach and to provide users with the appropriate working access. As shown in FIG. 24B, rescue towers 108-010 are also included in the set 100 in tray layer 130 which may be attached to the screw 10 after the removal of the integrated tower 20. These will allow access to the implants if further manipulation is necessary.

Preparation: The bony tissue preparation instruments are comprised of drill bits 108-013, 108-006, 108-007. These are well understood manual instruments that are commonly used for bony tissue removal and preparation. Each instrument connects to a standard ¼″ square quick connect handle 200, 300 which are also provided and shown in FIG. 24B.

Implant Delivery: The implant (screw) delivery instruments are comprised of a threaded driver assembly 108-008, self-retaining driver 108-011 shown in FIG. 24A and SI screws 108-DLL, and load dispersion caps 108-012-DD shown in FIG. 24C in tray layer 140.

Graft Delivery: The delivery instruments are comprised of a bone graft filler tube 108-015 and graft filler tamp 108-016 shown in FIG. 24B in tray layer 130.

Each are commonly used instruments that are well understood for their respective intended use.

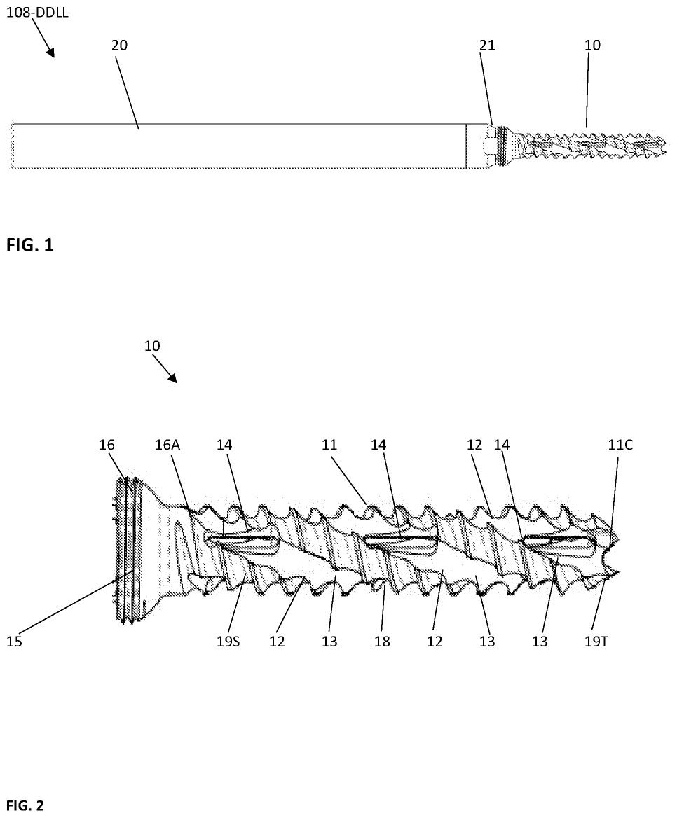

With reference to FIG. 1, the sacroiliac joint screw 108-DDLL of the present invention is shown with a tower 20 affixed to the head 15 of the screw 10. As shown, the tower 20 has a breakaway attachment 21 to allow removal from the screw 10.

FIG. 2 is a plan view of the sacroiliac joint screw 10 from FIG. 1 without the breakaway tower 20.

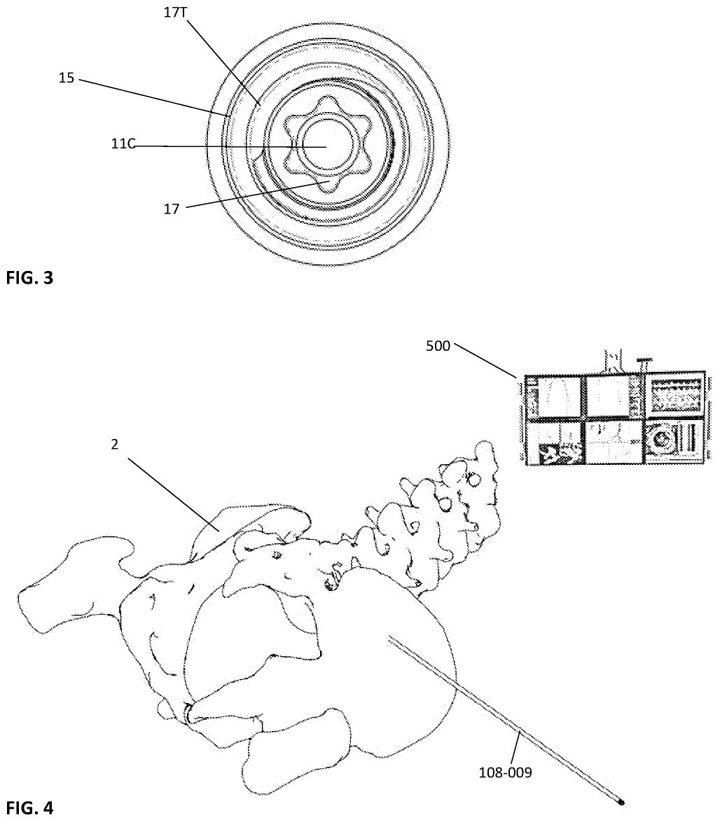

FIG. 3 shows a top view of the sacroiliac joint screw 10 of FIG. 2.

The sacroiliac joint screw 10 has a screw body 11 having a head 15 and a threaded shank 19 with a self-drilling cutting tip 19T. The screw body 11 is cannulated with a through hole 11C and configured to receive a Steinmann pin 108-009 for the purpose of a minimally invasive approach (MIS) for delivery to the sacroiliac joint (SI) while minimizing soft tissue damage. The self-drilling cutting tip 19T can create a pilot hole or enter a pre-drilled hole. The threaded shank 19 has threads 18 and a plurality of spiral cutting flutes 12, the spiral cutting flutes 12 extend along a length of the threaded shank 19 and are configured to provide a constant self-tapping feature. A plurality of bone harvesting windows 14 are positioned in a channel 13 in the spiral cutting flutes 12 and are configured to pull in bone as the screw 10 is advanced into the joint. As shown, the windows 14 are elongated slotted openings or holes to pass bone as the screw 10 is threaded into the SI bone 2.

The sacroiliac joint screw 108-DDLL as shown in FIG. 1, has an integrated removable tower 20 for soft tissue protection. The tower 20 is fixed or otherwise attached at a proximal end of the screw 10. The screw head 15 has a reverse threaded external head geometry 16 configured to allow a rescue tower 108-010 to attach to the screw 10 for screw removal. The reverse threaded external head geometry 16 is a left-handed thread which allows loosening of the screw 10. The screw head 15 has an undercut 16A on a distal side of the head. The undercut 16A allows for a stab and grab attachment to a driver. The screw 10 has a secure threaded post-fill bone graft delivery tube mechanism 17T to provide bone graft material.

As shown in FIG. 3, the sacroiliac joint screw 10 may further have a driver with a hexalobular drive configured to be received in an hexalobular drive aperture 17 in the screw head 15. The screw 10 is preferably made of a titanium alloy in diameter sizes of 8.0 mm to 12.0 mm and made in lengths from 25 mm to 60 mm.

The sacroiliac joint screw 10 may further include a load dispersion cap 108-012-DD, shown in FIG. 24, attached to an underside of the screw head 15. The cap 108-012-DD disperses the surface load and is configured to pivot and spin underneath the screw head 15.

A method of sacroiliac joint fusion comprises the steps of: localizing the sacroiliac joint 2 by fluoroscopy; inserting the 2.8 mm Steinmann Pin 108-009 to the plane of the SI joint; sliding Dilator One 108-002 over the Steinmann Pin 108-009; sliding the Dilator Two 108-003 over Dilator One 108-002; sliding Dilator Three 108-004 over Dilator Two 108-003; removing Dilator One 108-002 and inserting the Screw Depth Gauge 108-005 over the Steinmann Pin 108-009; reading the depth marking closest to the top of the Steinmann Pin 108-009; verifying the depth via fluoroscopy; removing the Screw Depth Gauge 108-005 and Dilator Two 108-003 to prepare for drilling; selecting the desired Drill Bit 108-013, 108-006, 108-007 size and drill to the depth reading indicated from the Screw Depth Gauge 108-005; attaching the appropriately sized SI Screw 108-DDLL to the physician's preferred Driver and insert over the Steinmann Pin 108-009; threading until fully seated; the Load Dispersion Cap 108-012-DD should be threaded over the distal end of SI Screw 10 prior to implantation if one is to be used removing Dilator Three 108-004 and inserting the 14 mm or 16 mm Parallel Pin Guide 108-014, 108-016 over the Steinmann Pin 108-009; using fluoroscopic imaging to align the Parallel Pin Guide 108-014, 108-016; inserting the second Steinmann Pin 108-009 and repeating the steps above for any remaining SI Screws 108-DDLL; threading in the Graft Filler Tube 108-015 to the SI Screw 108-DDLL and using the Graft Tamp 108-016 to deliver any allograft or autograft of the physician's choice to the SI Screw cannulation 11C; and removing all instrumentation and snapping the extension towers 20 off the SI screws 10 via a rocking motion.

With reference to FIG. 4 an illustration of step 1 of an exemplary sacroiliac joint fusion procedure using a 2.8 mm Steinmann Pin 108-009 in combination with fluoroscopic imaging 500 to determine initial trajectory to the SI joint 2 is shown.

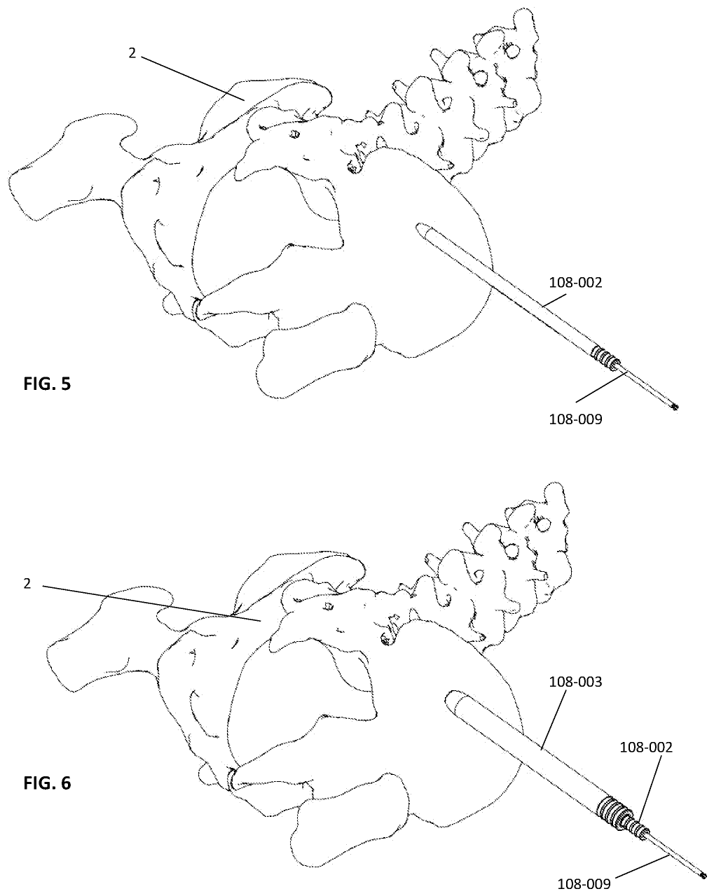

FIG. 5 shows step 2 of the procedure, Passing Dilator One 108-002 over the 2.8 mm Steinmann Pin 108-009. again, the surgeon may confirm trajectory using fluoroscopic imaging 500.

FIG. 6 shows step 3, Passing Dilator Two 108-003 over Dilator One 108-002. Again, the surgeon may confirm trajectory using fluoroscopic imaging 500.

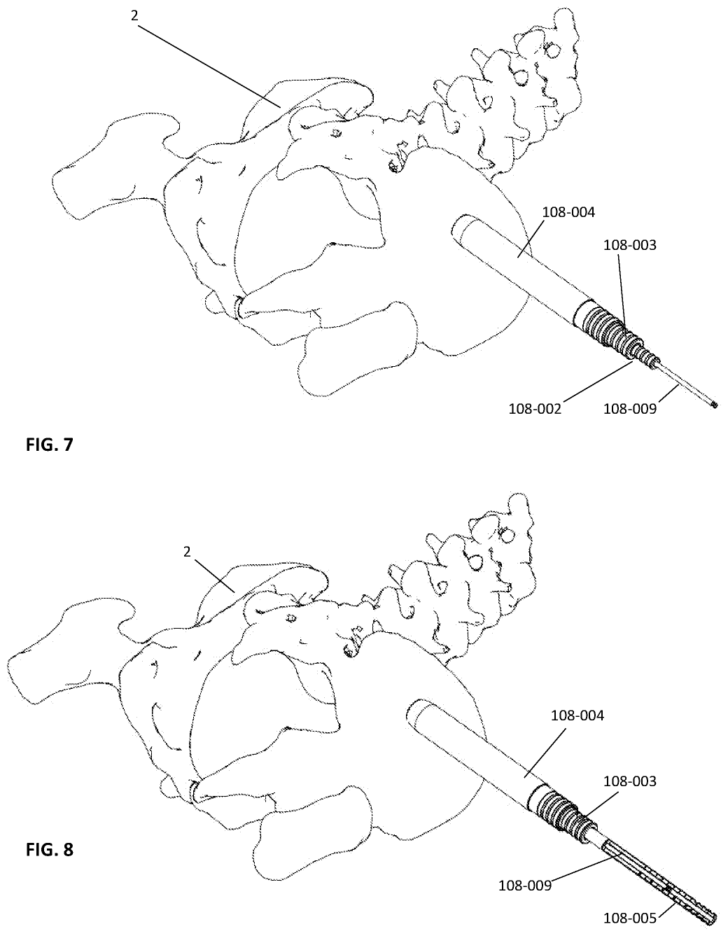

FIG. 7 shows step 4, Passing Dilator Three 108-004 over Dilator Two 108-003. Again, the surgeon may confirm trajectory using fluoroscopic imaging 500.

FIG. 8 shows step 5, Removing Dilator One 108-002 and inserting the Screw Depth Gauge 108-005 over the Steinmann Pin 108-009. Advancing the Depth Gauge 108-005 until it contacts bone distally. Read the closest number to the top of the Steinmann Pin 108-009 to measure the correct screw length.

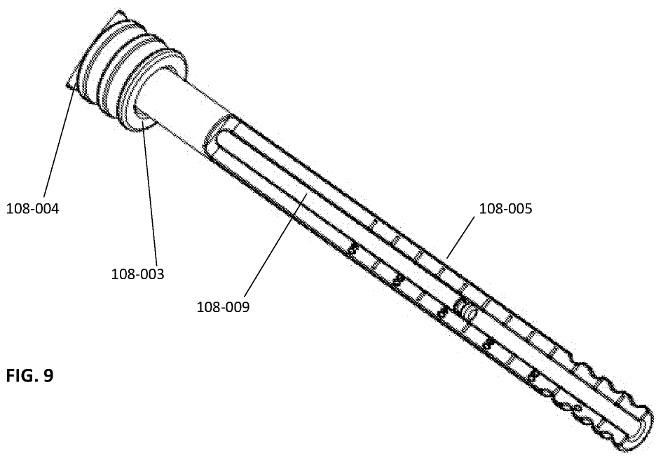

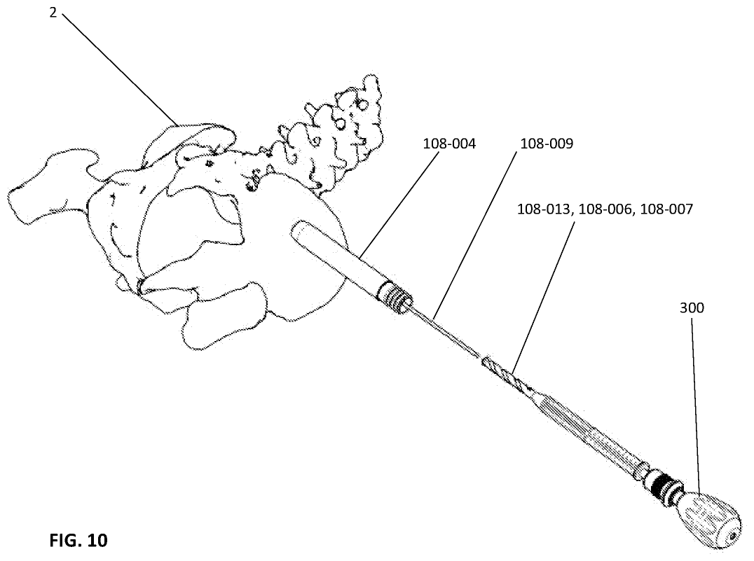

FIG. 9 shows step 6, Reading the closest number to the top of the Steinmann Pin 108-009. In this example 35 mm is the closest length number. Remove the Screw Depth Gauge 108-009 and Dilator Two 108-003. The Steinmann Pin 108-009 and Dilator Three 108-004 should remain in place, as shown in FIG. 10.

Once access has been gained connect the appropriately sized drill bit 108-013, 108-006 or 108-007, based on the preferred diameter of screw 10, to a quick connect handle 300 or drill motor. Drill bits are sized 3 mm smaller than the outer diameter of the SI Screw 10. Example, the 8 mm SI Screw 10 should utilize the 5 mm Drill Bit 108-013. Each Drill Bit is cannulated and designed to pass over the Steinmann Pin 108-009 and through Dilator Three 108-004. Each of the preparation instruments have sharp features at the distal working end and care should be taken during use and when exchanging instrumentation. Each of the preparation instruments are constructed from medical grade stainless steels and can be visualized on fluoroscopic imaging 500 if needed to confirm positioning.

FIG. 10 shows step 7, showing the 5, 7 or 9 mm Drill 108-0013, 108-006, 108-007 used with the Quick Connect Palm handle 300 or drill motor.

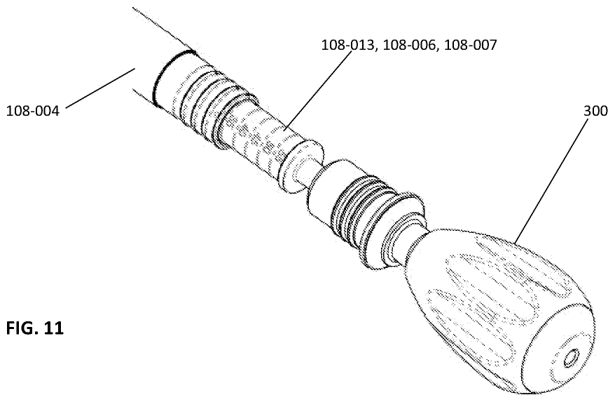

FIG. 11 shows step 8, advancing the Drill Bit 108-0013, 108-006, 108-007 to the corresponding number read from the Screw Depth Gauge 108-005.

Once the site has been prepared the delivery instruments can be used to Implant the SI Screw 10 and bone graft.

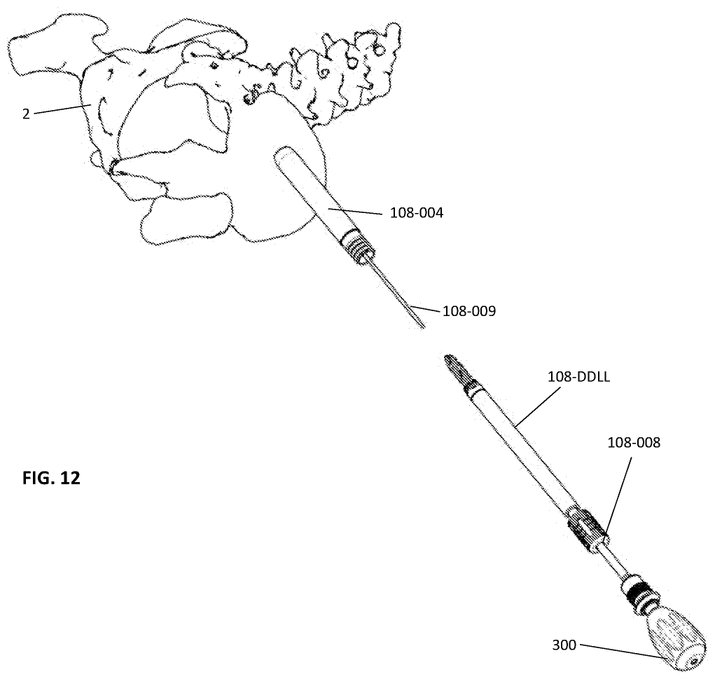

FIG. 12 shows step 9, threading the appropriately sized SI Screw 108-DDLL onto the Driver Assembly 108-008 until tight. At this time, a Load Dispersion Cap 108-012-DD may be threaded onto the end of the SI Screw 10, if preferred. Align the cannulation 11C of the SI Screw 10 with the Steinmann Pin 108-009. Alternatively, the Self-Retaining Driver 108-011 may be used in lieu of the Driver Assembly 108-008.

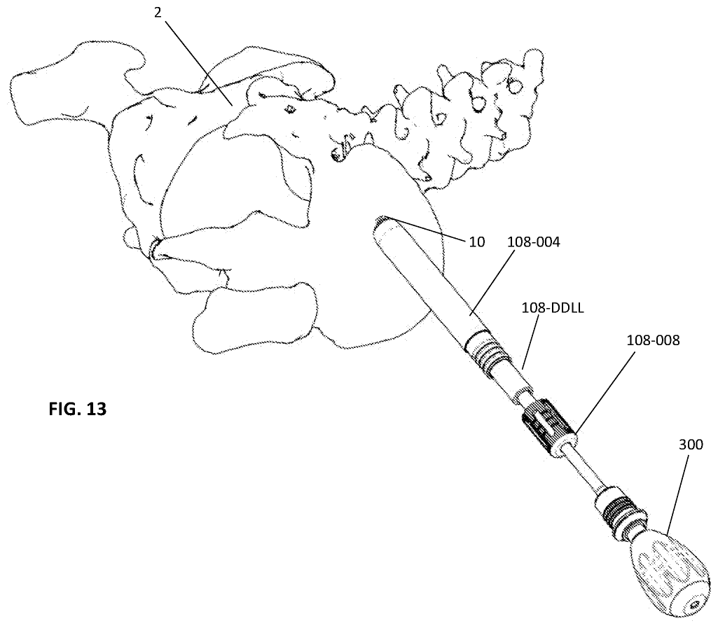

FIG. 13 shows step 10, Advancing the SI Screw 108-DDLL over the Steinmann Pin 108-009 and thread into the prepared bone hole until tight.

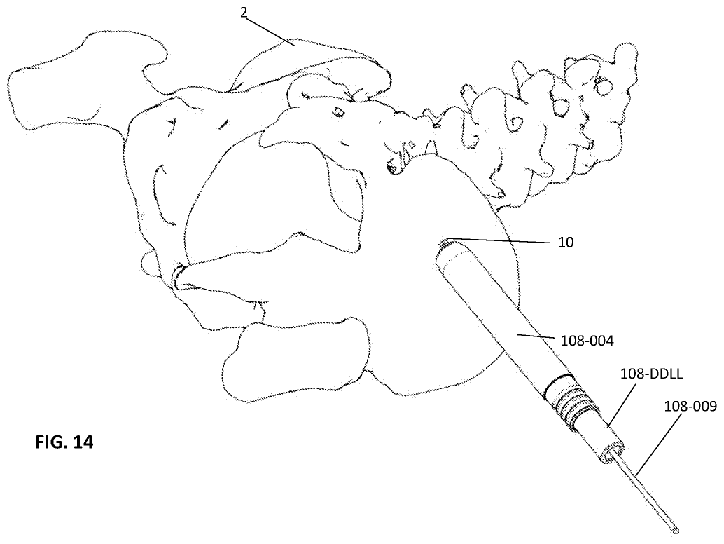

FIG. 14 shows step 11, Removing the Driver Assembly 108-008 by rotating the knurled knob handle 300 counterclockwise.

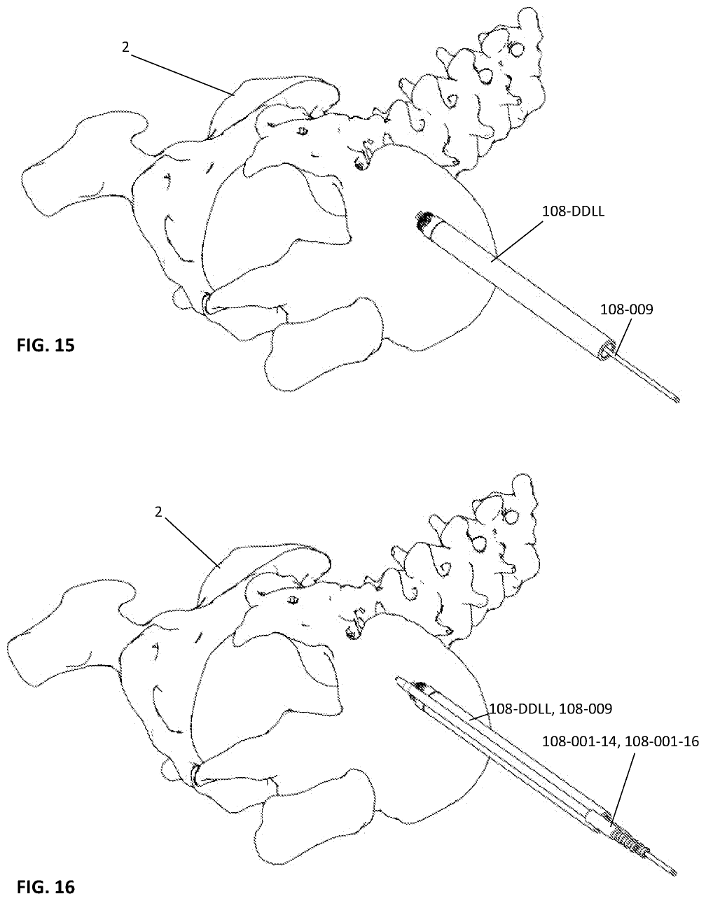

FIG. 15 shows step 12, Removing Dilator Three 108-004 leaving behind the Steinmann Pin 108-009.

FIG. 16 shows step 13, Placing the preferred sized Parallel Pin Guide 108-001-14, 108-001-16 over the Initial Steinmann Pin 108-009. Advance until it contacts bone 2. The Parallel Pin Guide comes in 14 mm 108-001-14 and 16 mm 108-001-16 center to center spacing.

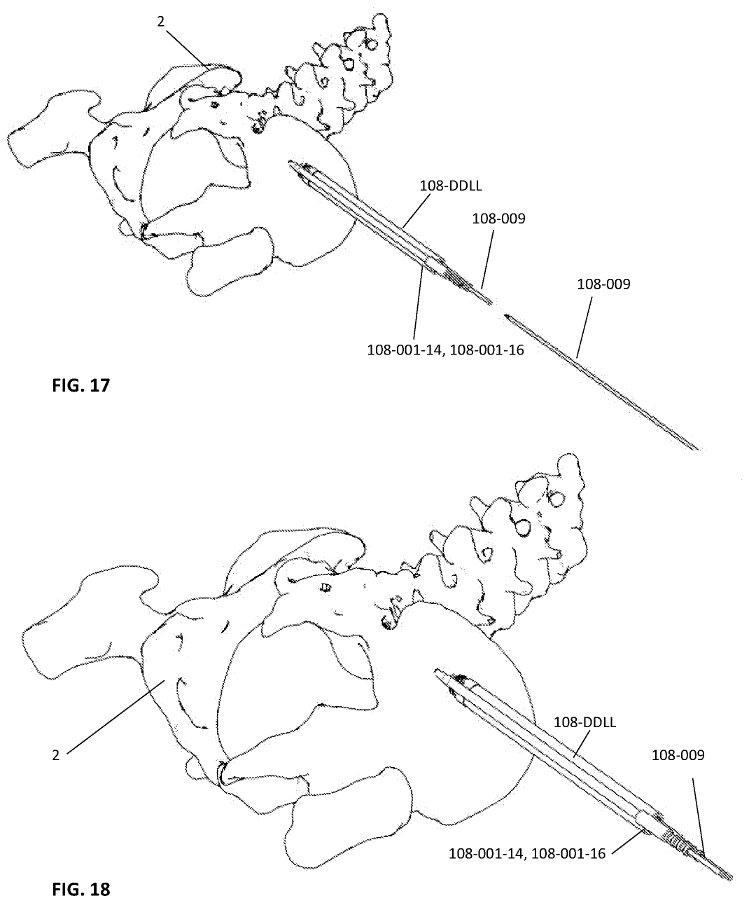

FIG. 17 shows step 14, Using fluoroscopic imaging 500 align the Parallel Pin Guide 108-001-14, 108-001-16 in the correct orientation and deliver a second Steinmann Pin 108-009 through the open cannula.

FIG. 18 shows step 15, Advance the second Steinmann Pin 108-009 to the desired depth. Then, Step 16 is Removing the Parallel Pin Guide 108-001-14, 108-001-16 and first Steinmann Pin 108-009 from the initial SI Screw 108-DDLL. Repeat steps 2 through 16 to place any additional SI Screws 108-DDLL in the joint 2.

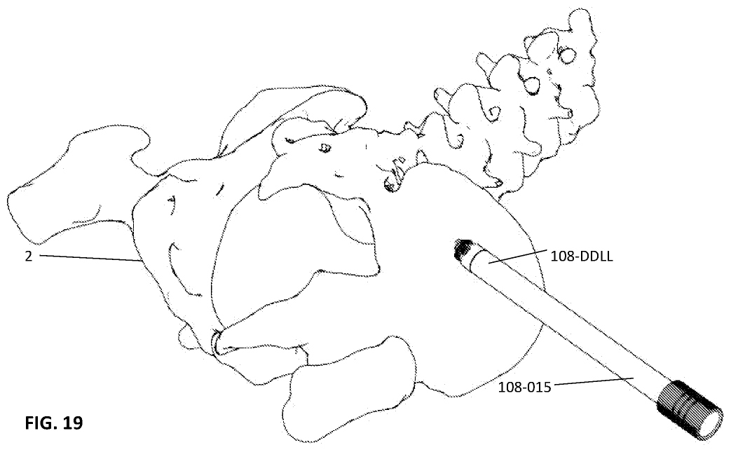

FIG. 19 shows step 17, Filling the Graft Filler Tube 108-015 with the surgeons preferred autograft or allograft. Note: The aperture of the cannulation 11C in the SI Screw is 3.0 mm. Large bone graft may not flow as intended if used. Insert the Graft Filler Tube 108-015 into the SI Screw tower 20 and thread until tight. A laser marked line on the Graft Filler Tube 108-015 will denote when it is fully seated.

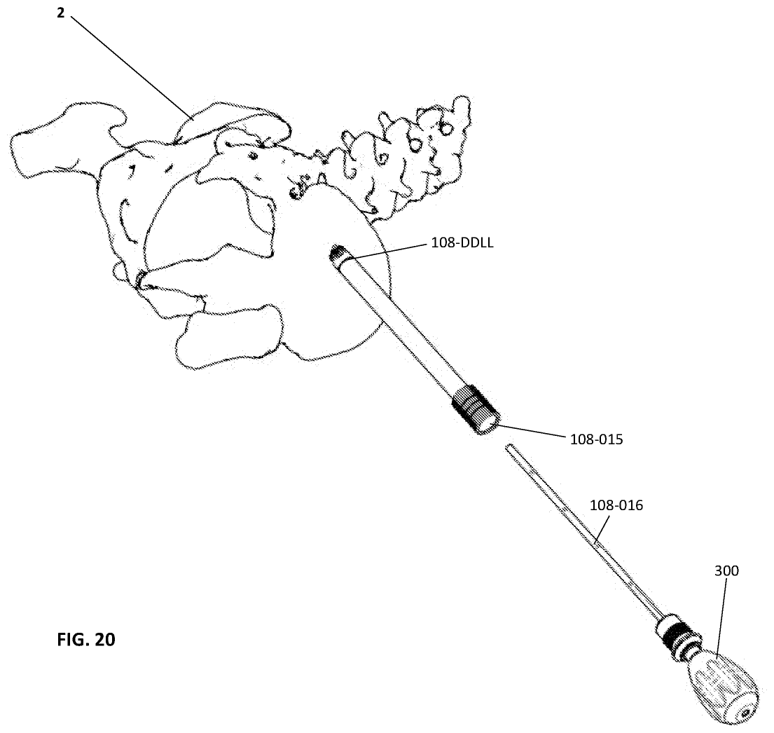

FIG. 20 shows step 18, Loading the Graft Filler Tamp 108-016 on the quick connect handle 300.

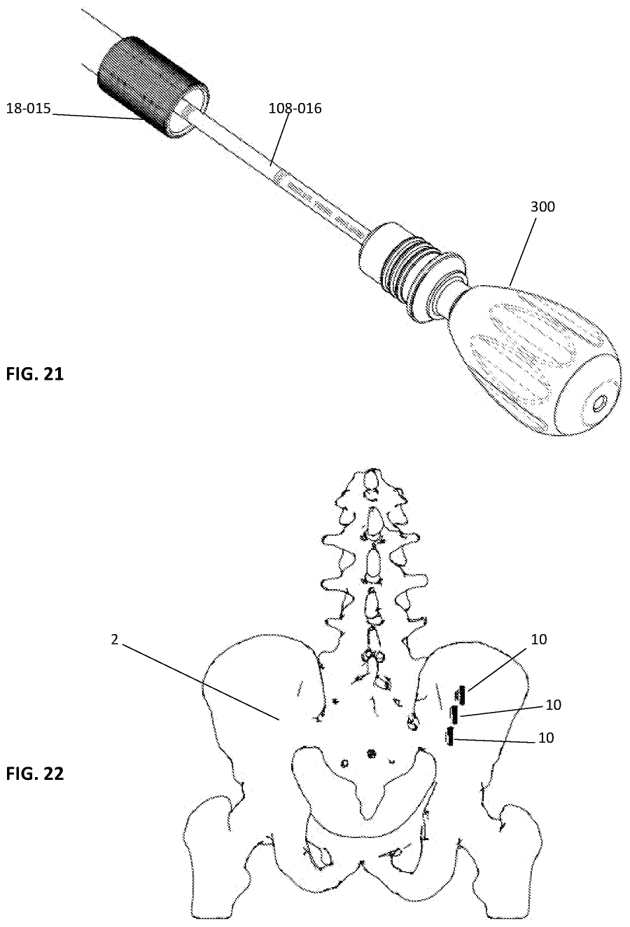

FIG. 21 shows step 19, Advancing the Tamp 108-016 deploying the bone graft into the SI Screw 10 and surrounding bone 2. The Tamp 108-016 is notated with 1 cc, 2 cc, and 3cc filler lines. Note: Repeat this process as much as needed to deliver the desired amount of bone graft.

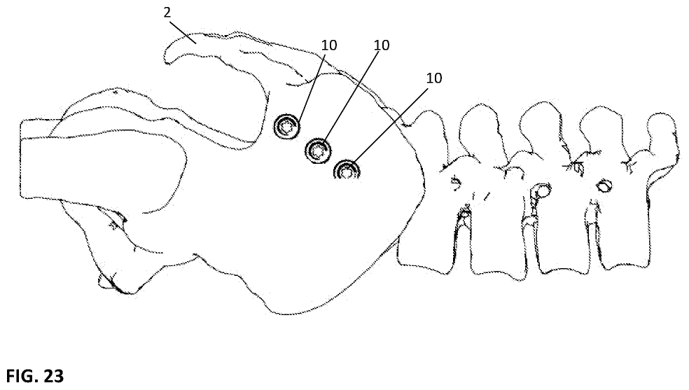

FIGS. 22 and 23 show step 20, after Removing the towers 20 from the SI Screws 10 by rocking them anterior to posterior causing them to release from the screw 10.

Once the operation has been determined to be complete, each of the instruments should be disassembled and returned to their respective locations in the instruments tray 110. An exemplary tray 110 with the instrumentation that is included in the system 100, and their respective intent for general use is shown in FIG. 24. In addition to the listed instruments, there are supporting off-the-shelf instruments such as ergonomic handles 200, 300 included in the set 100 as well.

The Sacroiliac Joint Fusion System consists of cannulated, fully threaded screws 10 with double helix threads 18 designed to be able to screw into pre-drilled bone 2. It is fabricated from medical grade titanium alloy, Ti-6A1-4V ELI (ASTM F-136). The system comes in various sizes and lengths to accommodate patient anatomy. Optional load bearing washers are included for each screw diameter to aid in conforming to patient anatomy. The Sacroiliac Joint Fusion System is comprised of various surgical instruments to be used to prepare the site to insert the system implants. All the instruments are made from surgical grade materials.

Safety Precautions: The SI Fusion Set should only be used by trained Physicians. The instrument set and implants should be reviewed with the provided literature prior to use until the physician is comfortable and familiar with the system. No special training, tools, software, power, or accessories are needed for the use of the system. If a user reviews the system prior to a surgery and is not familiar with use of a specific instruments or has not used one of the provided manual instruments, then extra time and care should be taken to ensure proper use. Each user is urged to first read all information contained in this insert. The use of an instrument for a task other than that for which it is intended, as well as improper, ineffective and insufficient maintenance can greatly reduce the life of an instrument and will invalidate the instruments warranty. Incorrect handling and care as well as misuse can lead to premature wear or can cause hazards to patients and users. The instruments and implants are supplied non-sterile and must be cleaned and sterilized prior to use according to hospital protocol and the procedures outlined in this document. Failure to follow these procedures will invalidate the instrument's warranty and can cause the instrument to fail.

Contraindications for use of the Sacroiliac Joint Fusion System include, but is not limited to: Infection, Tumor, Severe osteoporosis, Mental or physical impairments that limit a patient's ability to comply with necessary limitation of postoperative instructions, etc.

Cleaning and Maintenance: Test all instruments, accessories and equipment prior to each use. Written Standard Operating Procedures for the cleaning, sterilization, storage, inspection and maintenance of the instruments, accessories and equipment are recommended. Do not use in the presence of flammable liquids or anesthetics. Follow all safety precautions and instructions supplied by the manufacturer of the supplemental instruments. Failure to observe these cautions and contraindications may result in the injury, malfunction or other unanticipated occurrences or events for the operator, staff and/or the patient. Every surgical instrument should be disinfected and thoroughly cleaned after each use. Proper cleaning, inspection and maintenance will help ensure correct function of the surgical instrument. Clean, inspect and test each instrument carefully. Sterilize all instruments before surgery. A good cleaning and maintenance procedure will extend the useful life of the instrument. Special attention should be paid to slots, stops, ends, hollow tubes and other highly inaccessible areas. Check for cuts, voids, cracks, tears, abrasions, etc. Do not use damaged instruments. Cleaning and rinsing must take place immediately after each use for best effect. Failure to clean promptly may result in adherent particles or dried secretions that may resist cleaning and complicate or resist future sterilization. Instruments must be completely cleaned and rinsed of all foreign matter. Use warm water and a commercially available instrument pre-soak or cleaning agent. Enzymatic cleaners must be used to remove protein deposits. Follow the enzymatic cleaner's instructions, rinse thoroughly.

Do not use corrosive cleaning agents such as bleach. Cleaning solutions and rinses at or near a neutral pH (7.0) are best. Do not use abrasive cleaners. Only a soft bristle brush should be used. Can be disinfected in the washing machine up to 203 degrees F. (95 degrees C.). Rinse thoroughly with distilled water. Prepare for storage and/or sterilization.

After cleaning and rinsing, dry instruments completely and carefully with compressed air including inside channels and highly inaccessible areas. Note: After cleaning and before sterilization, treat all instruments with a water-soluble lubricant which is considered as being physiologically safe, especially their blades, ends, stops, snaps and all moveable parts.

Storage and Sterilization Instructions: Instruments must be stored in a clean, dry, moisture free area. The instruments should be stored individually in their shipping carton or in a protective tray with partitions. Protect tips with cloth, gauze or tubing if stored in drawers. Instruments are reusable and meet AAMI standards for sterilization. Use steam autoclave sterilization. Thoroughly clean instruments of all debris, tissue and foreign matter prior to sterilization. Follow the sterilizer manufacturer's instructions for operation and loading of steam autoclaves. There must be direct steam exposure to all surfaces of the instruments be sterilized including the internal surface of tubes and channels. Allow instrument to air cool to room temperature before use. Use steam autoclave sterilization only. Other time and steam temperature cycles may also be used. However, user must validate any deviation from the recommended time and temperature. (Note: Contact the manufacturer of the steam autoclave to confirm appropriate temperatures and sterilization times.) Caution: Autoclave temperatures should not exceed 279° F. (135° C.); handles, insulation or other non-metallic parts may be damaged. Do not sterilize with hot air.

An exemplary pre-vacuum cycle would be at temperature 270-275 degrees F., 132-135 degrees C. for a minimum exposure time of 4 minutes, minimum dry time of 20 minutes and minimum cool time of 30 minutes.

The dimensions shown in the kit and discussed in the detailed description are exemplary, it is understood other sizes and dimensions are within the scope of the invention.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A sacroiliac joint screw comprises:
a screw body having a head and a threaded shank with a self-drilling cutting tip, the screw body is cannulated and configured to receive a Steinmann pin for the purpose of a minimally invasive approach (MIS) for delivery to the sacroiliac joint (SI) while minimizing soft tissue damage.

2. The sacroiliac joint screw of claim 1 wherein the self-drilling cutting tip creates a pilot hole.

3. The sacroiliac joint screw of claim 1 wherein the threaded shank has a plurality of spiral cutting flutes, the spiral cutting flutes extend along a length of the threaded shank and are configured to provide a constant self-tapping feature.

4. The sacroiliac joint screw of claim 3 wherein a plurality of bone harvesting windows are positioned in the spiral cutting flutes configured to pull in bone as the screw is advanced into the joint.

5. The sacroiliac joint screw of claim 1 further comprises:
an integrated removable tower for soft tissue protection, the tower being fixed at a proximal end of the screw.

6. The sacroiliac joint screw of claim 1 wherein the screw head has a reverse threaded external head geometry configured to allow a rescue tower to attach to the screw for screw removal.

7. The sacroiliac joint screw of claim 6 wherein the reverse threaded external head geometry is a left-handed thread which allows loosening of the screw.

8. The sacroiliac joint screw of claim 1 wherein the screw head has an undercut on a distal side of the head, the undercut allows for a stab and grab attachment to a driver.

9. The sacroiliac joint screw of claim 1 wherein the screw has a secure threaded post-fill bone graft delivery tube mechanism to provide bone graft material.

10. The sacroiliac joint screw of claim 1 further comprises:
a driver with a hexalobular drive configured to be received in an hexalobular drive aperture in the screw head.

11. The sacroiliac joint screw of claim 1 wherein the screw is made of a titanium alloy.

12. The sacroiliac joint screw of claim 1 wherein the screw is made in diameter sizes of 8.0 mm to 12.0 mm.

13. The sacroiliac joint screw of claim 12 wherein the screw is made in lengths from 25 mm to 60 mm.

14. The sacroiliac joint screw of claim 1 further comprises:
a load dispersion cap attached to an underside of the screw head, the cap disperses the surface load and is configured to pivot and spin underneath the screw head.

15. A method of sacroiliac joint fusion comprises the steps of:
localizing the sacroiliac joint by fluoroscopy;
inserting a 2.8 mm Steinmann pin to the plane of the sacroiliac joint;
sliding dilator one over the Steinmann pin;
sliding dilator two over dilator one;
sliding dilator three over dilator two;
removing dilator one and inserting a screw depth gauge over the Steinmann pin;
reading a depth marking closest to the top of the Steinmann pin;
verifying the depth via fluoroscopy;
removing the screw depth gauge and dilator two to prepare for drilling;
selecting a desired drill bit size and drill to the depth reading indicated from the screw depth gauge;
attaching the appropriately sized sacroiliac screw to the physician's preferred driver and insert over the Steinmann pin;
threading until fully seated; a load dispersion cap should be threaded over the distal end of sacroiliac screw prior to threading/implantation if one is to be used;
removing dilator three and inserting a 14 mm or 16 mm parallel pin guide over the Steinmann pin;
using fluoroscopic imaging to align the parallel pin guide;
inserting a second Steinmann pin and repeating the steps above for any remaining sacroiliac screw;
threading in a graft filler tube to the sacroiliac screw and using a graft tamp to deliver any allograft or autograft of the physician's choice to the sacroiliac screw cannulation; and
removing all instrumentation and snapping the extension towers off the sacroiliac screws via a rocking motion.
